(19) United States
(12) Patent Application Publication
(10) Pub. No.: US 2014/0228753 A1
(43) Pub. Date: Aug. 14, 2014

(54) SPRING AUTO-DISABLE SYRINGE

(71) Applicant: Lingyang Huang, Shantou City (CN)

(72) Inventor: Lingyang Huang, Shantou City (CN)

(73) Assignees: SHANXI XINHUAMEI MEDICAL APPARATUS CO., LTD., Changzhi City, Shanxi (CN); Lingyang HUANG, Shantou City, Guangdong (CN)

(21) Appl. No.: 14/345,843

(22) PCT Filed: Sep. 12, 2012
(86) PCT No.: PCT/CN2012/081277

§ 371 Date: Mar. 19, 2014

(30) Foreign Application Priority Data
Sep. 20, 2011 (CN) 201110279759

(51) Int. Cl. A61M 5/50

(52) U.S. Cl. CPC A61M 5/50; A61M 2205/19; A61M 2205/273
USPC 604/110

(57) ABSTRACT

An auto-disable syringe is provided. The auto-disable syringe includes an injection tube connected to a needle seat and an injection needle connected to an auto-disable member. The auto-disable member connected to the injection needle is disposed on the needle seat. The injection tube is internally provided with a stopper and a hollow push tube. The push tube is internally provided with a spring seat accommodated by a spring and a stop block. A lower end of the auto-disable member is provided with a hook, and an upper end of the spring seat is provided with a hook groove for fastening with the hook. A cartridge is accommodated between the push tube and the spring seat. An upper part of the cartridge is accommodated by the stopper, and a lower part of the cartridge is abutted by one end of the spring in a compressed state.

### BACKGROUND OF THE INVENTION

a) Field of the Invention

The invention relates in general to a disposable medical equipment, and more particularly to an auto-disable syringe after being utilized.

b) Description of the Prior Art

Syringes are a common medical equipment for injecting a medicine into a human body to have the medicine come into direct contact with the human blood and fluid. To prevent infections and transmitted diseases, it is clearly stated by law that syringes are prohibited from repeated use and must be discarded after use. In reality, by exploiting monitor loopholes of medical wastes, there are certain offenders that trade used syringes in seeking for profits. One propagation means of the well-known fatal disease of the century, AIDS (Acquired immunodeficiency syndrome), is sharing and repeated use of syringes, bringing immense threat to people's health and well-being. Therefore, an auto-disable syringe, which automatically loses the injection capability once the injection by the syringe is complete, is later available. With the auto-disable syringe, a conventional push tube is designed as a hollow push tube, and an injection needle is pulled from an original position into the push tube by a member such as a spring, such that the syringe is disabled for further injection to prevent the syringe from repeated use. However, as certain problems exist among cooperative operations of the members in the design of current auto-disable syringes, it is a frequent occurrence that the injection needle cannot be pulled into the push tube after use. The above issue degrades the reliability of such conventional auto-disable syringes.

### SUMMARY OF THE INVENTION

It is an object of the present invention to provide a highly reliable spring auto-disable syringe capable of pulling an injection needle into a push tube after the syringe completes an injection.

According to an embodiment of the present invention, a spring auto-disable syringe includes an injection tube connected to a needle seat and an injection needle connected to an auto-disable member. The auto-disable member connected to the injection needle is disposed on the needle seat. The injection tube is internally provided with a stopper and a hollow tube. The push tube is provided with a spring seat accommodated by a spring and a stop block. A lower end of the auto-disable member is provided with a hook. An upper end of the spring seat is provided with a hook groove for correspondingly fastening with the hook. The spring auto-disable syringe of the present invention is characterized that, a cartridge is accommodated between the push tube and the spring seat, an upper part of the cartridge is accommodated by the stopper, and a lower part of the cartridge is abutted against by one end the spring in a compressed state.

In one embodiment, the hook at the lower end of the auto-disable member is uncompressible and non-deformable, and the hook groove at the upper end of the spring seat is provided with a notch.

In one embodiment, the hook groove at the upper end of the spring seat is internally provided with a slanted platform.

In one embodiment, a horizontal included angle of the slanted platform is 20 to 85 degrees.

In one embodiment, an upper end of the cartridge is provided with a hedge for accommodating and fastening with the stopper.

In one embodiment, a lower end of the cartridge is provided with a hedge for abutting against the spring in a compressed state.

According to another embodiment of the present invention, a spring auto-disable syringe includes an injection tube connected to a needle seat and an injection needle connected to an auto-disable member. The auto-disable member connected to the injection needle is disposed on the needle seat. The injection tube is internally provided with a stopper and a hollow push tube. The push tube is internally provided with a spring seat accommodated by a spring and a stop block. The spring auto-disable syringe of the present invention is characterized that, an upper end of the spring seat is provided with a hook, a lower end of the auto-disable member is provided with a hook groove for correspondingly fastening with the hook, a cartridge is accommodated between the push tube and the spring seat, an upper part of the cartridge is accommodated and fastened with a stopper, and a lower part of the cartridge is abutted against by one end of the spring in a compressed state.

In one embodiment, the upper end of the cartridge is provided with a hedge for fastening with the stopper, and a lower end of the cartridge is provided with a hedge for abutting against the spring in a compressed state.

In one embodiment, the hook groove at the lower end of the auto-disable member is provided with a notch, and the hook at the upper end of the spring seat is uncompressible and non-deformable.

In one embodiment, the hook groove at the upper end of the spring seat is internally provided with a slanted platform, and a horizontal included angle of the slanted platform is 20 to 85 degrees.

Thus, the members of the present invention are operable in coordination with one another, so that the injection needle can be pulled into the push tube after completing an injection, thereby offering high reliability with a simple structure. Meanwhile, the lower end of the auto-disable member is designed as a hook groove and the upper end of the spring seat is designed as a hook for corresponding fastening, thereby also achieving the technical object of the present invention.

The above and other aspects of the invention will become better understood with regard to the following detailed description of the preferred but non-limiting embodiments. The following description is made with reference to the accompanying drawings.

### BRIEF DESCRIPTION OF THE DRAWINGS

FIG. 1 is a schematic diagram of the present invention;

FIG. 2 is a schematic diagram of the present invention applied to a first injection;

FIG. 3 is a schematic diagram of a spring seat connected to an auto-disable member provided with an injection needle of the present invention;

FIG. 4 is a schematic diagram of a stopper abutting against a top end of an injection tube of the present invention;

FIG. 5 is a schematic diagram of a push tube being continued pushed upward in a way that a spring seat passes through a stop block in the push tube and jointly pushed into the push tube with an injection needle of the present invention;

FIG. 6 is a schematic diagram of a cartridge of the present invention;

FIG. 7 is a sectional view of a hook groove at an upper end of a spring seat of the present invention; and

FIG. 8 is a schematic diagram of a hook groove provided with a slanted platform at an upper end of a spring seat of the present invention.

### DETAILED DESCRIPTION OF THE INVENTION

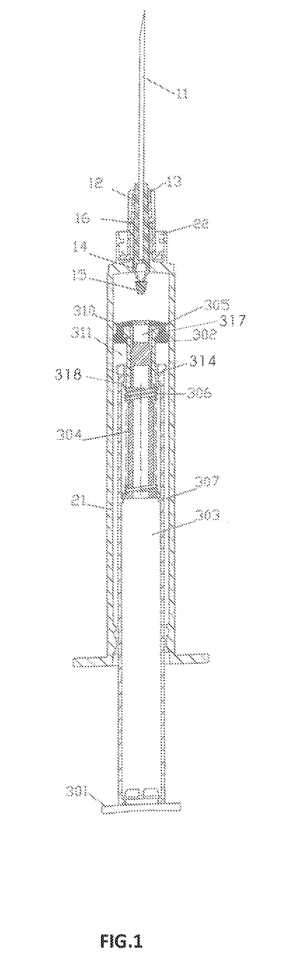

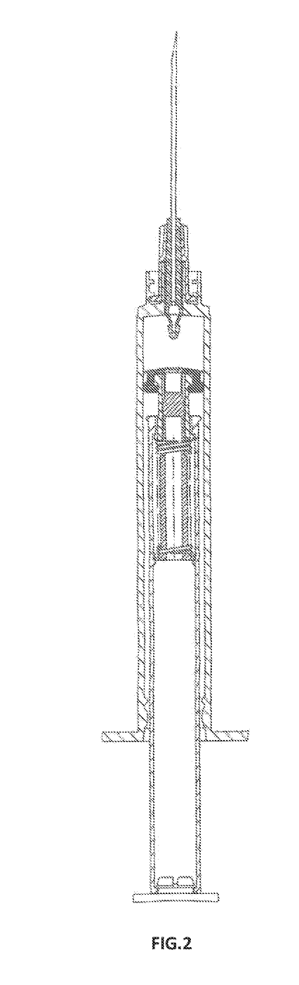

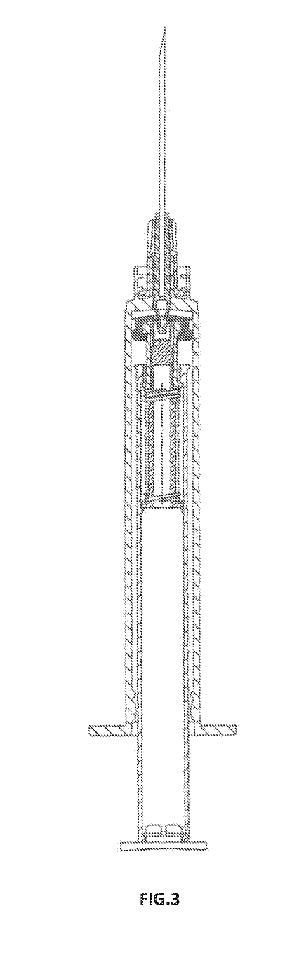

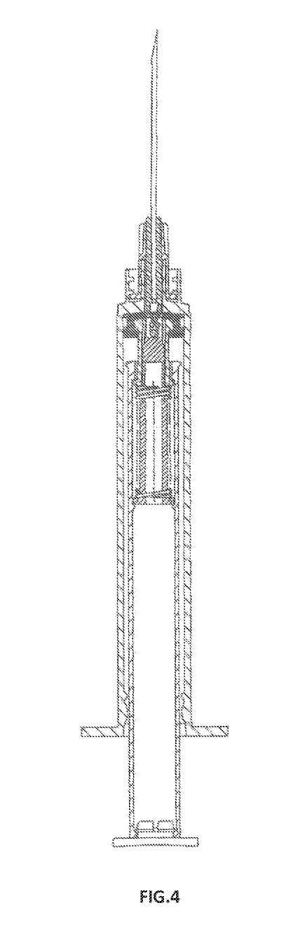

Referring to FIGS. 1 to 5, a cartridge 314 is utilized as a connecting member between a push tube 301 and a spring seat 304. The push tube 301 is a hollow body that at the same time accommodates a stopper 302. An upper end of the cartridge 314 is provided with a hedge 317 for fastening with the stopper 302. After completing an injection, the stopper 302 is pushed to a top portion 22 of the injection tube 21 by the push tube 301, and a hook 15 of an auto-disable member 12 is fastened with a hook groove 305 of the spring seat 304. That is, the spring seat 304 is connected with the auto-disable member 12, and the stopper 302, the cartridge 314 and the spring seat 304 are all halted from upward movements. At this point, the push tube 301 is still pushed upward, a spring 306 on the spring seat 304 is compressed, and an upper end of the compressed spring 306 is abutted against a lower end of the cartridge 314. A lower end of the cartridge 314 is provided with a spring seat ledge 318 for abutting against the spring 306 is a compressed state, and the spring seat 304 is abutted by a stop block 307 in the push tube 301. As such, the force thrusting the push tube 301 upward is greater than the force of the stop block 307 that abuts the compressed spring 306, and so the spring seat 304 passes through the stop block 307, and the spring 306 restores an original state and has the upper end abutting against the cartridge 314. During the restoring process of the spring 306, the spring seat 304 is pulled back into the push tube 301, and the spring seat 304 then becomes again connected to the auto-disable member 12 disposed with an injection needle 11. Thus, the injection needle 11, the auto-disable member 12 and the spring seat 304 are pulled into the push tube 301 to disengage the injection needle 11 from a position originally placed with the needle seat 13 and to disable the syringe from another injection, thereby achieving the object of auto-disabling after use of the present invention. Therefore, in the present invention, the injection needle 11 is successfully pulled into the push tube 301 after an injection is performed, thereby offering high reliability with a simple structure.

Meanwhile, the lower end of the auto-disable member 12 is designed as a hook groove 305 and the upper end of the spring seat 304 is designed as a hook 15 for corresponding fastening, thereby also achieving the technical object of the present invention.

A spring auto-disable syringe including an injection tube 21 connected to a needle seat 13, and an injection needle 11 connected to an auto-disable member 12. The auto-disable member 12 connected to the injection needle 11 is disposed on the needle seat 13, the injection tube is internally provided with a stopper and a hollow push tube. The push tube is internally provided with a spring seat accommodated by a spring, and a stop block. The spring auto-disable syringe is characterized that, an upper end of the spring seat is provided with a hook, a lower end of the auto-disable member is provided with a hook groove for fastening with the hook, a cartridge is accommodated between the push tube and the spring seat, an upper part of the cartridge is accommodated by the stopper, and a lower part of the cartridge is abutted by one end of the spring in a compressed state.

An upper end of the cartridge is provided with a hedge for fastening with the stopper, and a lower end of the cartridge is provided with a hedge for abutting against the spring in a compressed state.

The hook groove at the upper end of the spring seat is provided with a notch, the hook at the lower end of the auto-disable member is uncompressible and non-deformable, and the.

The hook groove at the upper end of the spring seat is internally provided with a slanted platform, and the slanted platform has a horizontal included angle of 20 to 85 degrees.

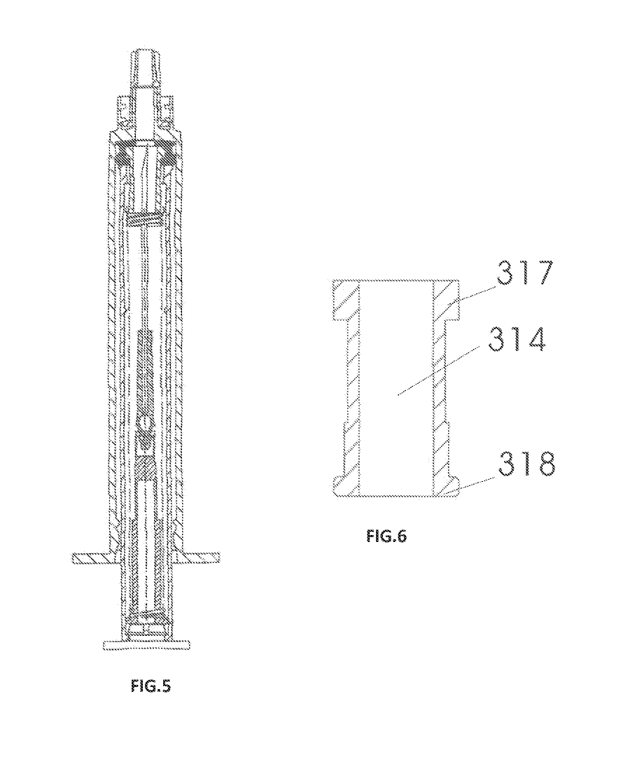

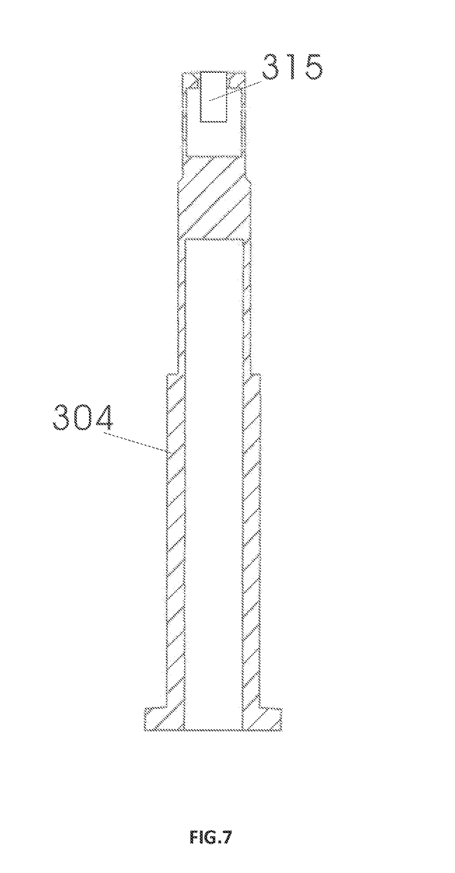

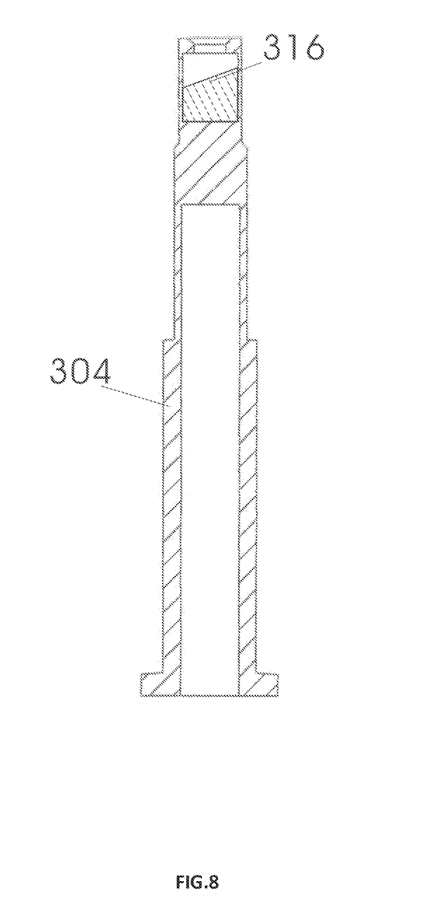

Referring to FIGS. 1 to 8, it is seen from an operation process of the present invention that, the cartridge 314 is utilized as a connecting member between the push tube 301 and the spring seat 304, with the cartridge 314 at the same time accommodating and connecting the stopper 302. After completing an injection, the stopper 302 is pushed to the top portion 22 of the injection tube 21 by the push tube 301, and the hook 15 of the auto-disable member 12 is fastened with the hook groove 305 of the spring seat 304. That is, the spring seat 304 is connected with the auto-disable member 12, and the stopper 302, the cartridge 314 and the spring seat 304 are all halted from upward movements. At this point, the push tube 301 is still pushed upward, the spring 306 on the spring seat 304 is compressed, the upper end of the compressed spring 306 is abutted against the lower end of the cartridge 314, the lower end of the compressed spring 306 is abutted against the spring seat ledge 318, and the spring seat ledge 318 is abutted by the stop block 313 in the push tube 301. As such, the force thrusting the push tube 301 upward is greater than the force of the stop block 313 that abuts the compressed spring 306, and so the spring seat 304 passes through the stop block 313, and the spring 306 restores an original state and has the upper end abut against the cartridge 314. During the restoring process of the spring 306, the spring seat 304 is pulled back into the push tube 301, and the spring seat 304 then again becomes connected to the auto-disable member 12 disposed with the injection needle 11. Thus, the injection needle 11, the auto-disable member 12 and the spring seat 304 are pulled into the push tube 301 to disengage the injection needle 11 from the position originally placed with the needle seat 13 and to disable the syringe from another injection, thereby achieving the object of auto-disable after use of the present invention. Therefore, in the present invention, the injection needle 11 is successfully pulled into the push tube 301 after an injection is performed, thereby offering high reliability with a simple structure. Meanwhile, the lower end of the auto-disable member 12 is designed as the hook groove 305 and the upper end of the spring seat 304 is designed as the hook 15 for corresponding fastening, thereby also achieving the technical object of the present invention.

As shown in FIGS. 1 to 8, the hook 15 at a lower end 14 of the auto-disable member 12 is uncompressible and non-deformable, and the hook groove 305 at the upper end of the spring seat 304 is provided with a notch 315. Due to the material made, usually polypropylene (PP), the notch 315 is provided with moderate elasticity such that the notch 315 can be slightly stretched open when the hook 15 is extended into the hook groove 305, thereby fastening the hook groove 305 with the hook 15. As the hook groove 305 at the upper end of the spring seat 304 is provided with a slanted platform 316, the auto-disable member 12 and an injection needle therein are slanted toward the injection tube 21. Thus, in an occasional event that the injection needle is not withdrawn into the push tube 301, the auto-disable member 12 and the injection needle are ensured to be pulled into the injection tube 21 instead of again pushing the injection needle out of the injection tube 21, thereby guaranteeing the prevention of the used syringe from a hazardous application. The slanted platform 316 has a horizontal included angle of 20 to 85 degrees.

While the invention has been described by way of example and in terms of the preferred embodiments, it is to be understood that the invention is not limited thereto. On the contrary, it is intended to cover various modifications and similar arrangements and procedures, and the scope of the appended claims therefore should be accorded the broadest interpretation so as to encompass all such modifications and similar arrangements and procedures.

1. A spring auto-disable syringe, including an injection tube connected to a needle seat and an injection needle connected to an auto-disable member;
auto-disable member connected to the injection needle being disposed on the needle seat, the injection tube being internally provided with a stopper and a hollow push tube, the push tube being internally provided with a spring seat accommodated by a spring and a stop block, a lower end of the auto-disable member being provided with a hook, an upper end of the spring seat being provided with a hook groove for fastening with the hook; wherein the spring auto-disable syringe comprising
a cartridge is accommodated between the push tube and the spring seat, an upper part of the cartridge is accommodated by the stopper, and a lower part of the cartridge is abutted by one end of the spring in a compressed state.

2. The spring auto-disable syringe according to claim 1, wherein the hook at the lower end of the auto-disable member is uncompressible and non-deformable, and the hook groove at the upper end of the spring seat is provided with a notch.

3. The spring auto-disable syringe according to claim 1, wherein the hook groove at the upper end of the spring seat is internally provided with a slanted platform.

4. The spring auto-disable syringe according to claim 3, wherein the slanted platform has a horizontal included angle of 20 to 85 degrees.

5. The spring auto-disable syringe according to claim 1, wherein an upper end of the cartridge is provided with a hedge for fastening with the stopper.

6. The spring auto-disable syringe according to claim 1, wherein a lower end of the cartridge is provided with a hedge for abutting against the spring in a compressed state.

7. A spring auto-disable syringe, including an injection tube connected to a needle seat and an injection needle connected to an auto-disable member; the auto-disable member connected to the injection needle being disposed on the needle seat, the injection needle being internally provided with a stopper and a hollow push tube, the push tube being internally provided with a spring seat accommodated by a spring and a stop block; wherein the spring auto-disable syringe comprising
an upper end of the spring seat is provided with a hook, a lower end of the auto-disable member is provided with a hook groove for fastening with the hook, a cartridge is accommodated between the push tube and the spring seat, an upper part of the cartridge is accommodated by a stopper, and a lower part of the cartridge is abutted by one end of the spring in a compressed state.

8. The spring auto-disable syringe according to claim 7, wherein an upper end of the cartridge is provided with a hedge for fastening with the stopper, and a lower end of the cartridge is provided with a hedge for abutting against the spring in a compressed state.

9. The spring auto-disable syringe according to claim 7, wherein the lower end of the auto-disable member is provided with a notch, and the hook at the upper end of the spring seat is uncompressible and non-deformable.

10. The spring auto-disable syringe according to claim 7, wherein the hook groove lower end of the auto-disable member is internally provided with a slanted platform, and the slanted platform has a horizontal included angle of 20 to 85 degrees.
